(19) United States
(12) Patent Application Publication
(10) Pub. No.: US 2011/0053290 A1
(43) Pub. Date: Mar. 3, 2011

(54) LIGHT ADDRESSING BIOSENSOR CHIP AND METHOD OF DRIVING THE SAME

(75) Inventors: Chang Geun Ahn, Daejeon (KR); Chan Woo Park, Daejeon (KR); Jong Heon Yang, Daejeon (KR); Tae Youb Kim, Seoul (KR); Chil Seong Ah, Daejeon (KR); An Soon Kim, Daejeon (KR); Bong Kyu Kim, Daejeon (KR); Gun Yong Sung, Daejeon (KR); Seon Hee Park, Daejeon (KR)

(73) Assignee: Electronics and Telecommunications Research Institute, Daejeon (KR)

(21) Appl. No.: 12/858,158

(22) Filed: Aug. 17, 2010

(30) Foreign Application Priority Data
Aug. 28, 2009 (KR) 10-2009-0080443

(51) Int. Cl. G01N 33/566; H01L 29/772

(52) U.S. Cl. 436/501; 257/253; 257/E29.242

(57) ABSTRACT

Provided is a biosensor chip. The biosensor chip includes a plurality of biosensor cells that are arranged in a matrix and selectively generate and output a sensed signal by addressing of external light, at least one sensing line that is simultaneously connected with the plurality of biosensor cells and transmits the sensed signal from one selected from the biosensor cells, and an output terminal that receives the sensed signal from the sensing line and outputs the sensed signal to an external reader. Thus, the biosensor cells are set in array in the biosensor chip without a separate driving unit, so that a process of manufacturing the biosensor chip is simplified. The biosensor cell to be sensed is selectively addressed through the external light, so that it is possible to reduce a price of the biosensor chip used as a disposable chip.

### CROSS-REFERENCE TO RELATED APPLICATION

This application claims priority to and the benefit of Korean Patent Application No. 10-2009-0080443, filed Aug. 28, 2009, the disclosure of which is incorporated herein by reference in its entirety.

### BACKGROUND

1. Field of the Invention

The present invention relates to a biosensor and, more particularly, to a biosensor to which light is addressed.

2. Discussion of Related Art

Recently, efforts have rapidly been made to develop nano-bio fusion technology that fuses nano-technology with bio-technology. Particularly, in a nano-bio chip field that is part of the nano-bio fusion technology, studies have been intensively made on biosensors aiming at detecting protein from blood.

Typically, silicon-based biosensors that can be mass-produced using semiconductor processes have been proposed. For example, as in FIG. 1, biosensor chip technology using a semiconductor micro-processing technique has been proposed.

The biosensor chip of FIG. 1 includes a biocell, in which specified probe molecules are distributed, using the structure of a conventional memory cell such as a dynamic random access memory (DRAM), directs a reaction with target molecules, and detects whether or not a corresponding biosensor cell reacts through a general addressing method used in the memory.

This biosensor chip includes a biosensor connected with a transistor. When the transistor is selectively turned on according to an addressing input signal from the outside, the biosensor chip outputs a detected signal of the connected biosensor.

To this end, the biosensor chip includes a row address unit and a column address unit connected with a biosensor array as in FIG. 1. Each of the row and column address units includes a buffer receiving an external address input signal, and a decoder outputting the address input signal to a corresponding address line. A circuit of this address unit may be formed together when the transistor of the biosensor cell is formed, or be attached by a separate chip after the biosensor is formed on a substrate.

However, when the address unit circuit for generating and outputting a detected signal is formed within the biosensor chip as in FIG. 1, a process of manufacturing the biosensor chip is complicated, and a cost of production of the biosensor chip disposed after being used once is increased.

### SUMMARY OF THE INVENTION

The present invention is directed to a biochip capable of moving magnetic particles effecting an antigen-antibody reaction in a small quantity of fluid stopped in a micro-channel using a magnetic force, and repeating mixing and cleaning processes for the antigen-antibody reaction to make a quantitative analysis of a trace of a target material within a short time.

An aspect of the present invention is to provide a biosensor chip, which includes: a plurality of biosensor cells having photoelectric elements arranged in a matrix, and selectively turned on to generate a reference electric signal by addressing of external light and biosensors receiving the reference electric signal, and generating and outputting a sensed signal caused by a reaction between probe molecules and target molecules on the basis of the reference electric signal; at least one sensing line simultaneously connected with the plurality of biosensor cells, and transmitting the sensed signal from one selected from the biosensor cells; and output terminal receiving the sensed signal from the sensing line, and outputting the sensed signal to an external reader.

In exemplary embodiments, the biosensor may have resistance changed by the reaction between the probe molecules and the target molecules.

In exemplary embodiments, the photoelectric element may includes: a solar cell generating a turn-on voltage by the addressing of the external light; and a transistor turned on by the turn-on voltage of the solar cell and transmitting the reference electric signal to the biosensor.

In exemplary embodiments, the transistor may includes: a gate electrode connected with the solar cell and receiving the turn-on voltage; a source electrode connected with a reference voltage; and a drain electrode connected with the biosensor and transmitting the reference electric signal based on the reference voltage.

In exemplary embodiments, the photoelectric element may include a phototransistor, which is turned on by the addressing of the external light and transmits the reference electric signal to the biosensor.

In exemplary embodiments, the phototransistor may include a semiconductor layer, from which electron-hole pairs are generated to reduce resistance by the addressing of the external light.

In exemplary embodiments, the phototransistor may include: a gate electrode connected with a first reference voltage a source electrode connected with a second reference voltage; and a drain electrode connected with the biosensor and transmitting the reference electric signal based on the first and second reference voltages.

In exemplary embodiments, the plurality of biosensor cells may be grouped into a plurality of biosensor cell groups, and one of the sensing lines may be simultaneously connected with the plurality of biosensor cells belonging to one of the biosensor cell groups.

In exemplary embodiments, the output terminals may be equal in number to the sensing lines.

In exemplary embodiments, the biosensor chip may further include a power supply terminal, which receives an external supply voltage to apply to the plurality of biosensor cells.

In exemplary embodiments, the probe molecules of each biosensor cell may be different from each other.

Another aspect of the present invention is to provide a biosensor chip, which includes: a plurality of biosensor cells arranged in a matrix, and selectively generating and outputting a sensed signal by addressing of external light; at least one sensing line simultaneously connected with the plurality of biosensor cells, and transmitting the sensed signal from one selected from the biosensor cells; and an output terminal receiving the sensed signal from the sensing line, and outputting the sensed signal to an external reader.

In exemplary embodiments, each biosensor cell may include: a photodiode having a p-type doping layer, an n-type doping layer, and a non-doping region; and a plurality of probe molecules immobilized on the non-doping region.

In exemplary embodiments, the photodiode may cause current to be changed by a change in transmittance caused by a reaction between the probe molecules and target molecules.

In exemplary embodiments, the biosensor chip may further include a power supply terminal, which receives an external supply voltage to apply to the plurality of biosensor cells.

In exemplary embodiments, the probe molecules of each biosensor cell may be different from each other.

Yet another aspect of the present invention is to provide a method of driving a biosensor chip, which includes: exposing a plurality of biosensor cells arranged in a matrix to a detection sample having target molecules; selecting at least one of the biosensor cells which is intended to generate and output a sensed signal, and addressing external light to the selected biosensor cell; turning on a transistor of the selected biosensor cell with the light, and outputting a reference electric signal to a biosensor of the selected biosensor cell; and generating the sensed signal from the biosensor based on the reference electric signal, and outputting the sensed signal to an output terminal.

In exemplary embodiments, the sensed signal of the biosensor cell may be transmitted to the output terminal through a sensing line connected with the plurality of biosensor cells at the same time.

In exemplary embodiments, each biosensor cell may include: a solar cell generating a turn-on voltage with the external light; a transistor turned on by the turn-on voltage of the solar cell and transmitting the reference electric signal; and a biosensor receiving the reference electric signal of the transistor to generate the sensed signal changed by a reaction between probe molecules and target molecules.

In exemplary embodiments, the each biosensor cell may include: a phototransistor having a semiconductor layer from which electron-hole pairs are generated by the external light to reduce resistance, turned on by the external light, and transmitting the reference electric signal; and a biosensor receiving the reference electric signal of the phototransistor to generate the sensed signal changed by a reaction between probe molecules and target molecules.

### BRIEF DESCRIPTION OF THE DRAWINGS

The above and other features and advantages of the present invention will become more apparent to those of ordinary skill in the art by describing in detail preferred embodiments thereof with reference to the attached drawings in which:

FIG. 1 illustrates the configuration of a conventional biosensor chip;

FIG. 2 illustrates the configuration of a biosensor chip according to an exemplary embodiment of the present invention;

FIG. 3 is a circuit diagram illustrating a biosensor cell according to a first exemplary embodiment of the present invention;

FIG. 4 is a cross-sectional view illustrating a biosensor cell according to a second exemplary embodiment of the present invention;

FIG. 5 is a cross-sectional view illustrating a biosensor cell according to a third exemplary embodiment of the present invention;

FIG. 6 is a graph illustrating sensed signals caused by a reaction of the biosensor cell of FIG. 5; and

FIG. 7 illustrates a biosensor chip and a light addressor in accordance with an exemplary embodiment of the present invention.

### DETAILED DESCRIPTION OF EXEMPLARY EMBODIMENTS

The present invention will be described more fully hereinafter with reference to the accompanying drawings, in which exemplary embodiments of the invention are shown. This invention may, however, be embodied in different forms and should not be construed as limited to the embodiments set forth herein. In the following description of the present invention, a detailed description of known functions and components incorporated herein will be omitted when it may make the subject matter of the present invention rather unclear. It should be noted that the same reference numbers are used in the figures to denote the same elements.

It will be understood that, throughout the specification, when an element is referred to as being “connected” or “coupled” to another element, it can be “directly connected” or “directly coupled” to the other element or “electrically connected” to the other element via at least one intervening element.

It will be understood that, throughout the specification, unless explicitly described to the contrary, the term “comprise” and its conjugations such as “comprises” or comprising” should be interpreted to include stated elements but not exclude any other elements. In addition, the term “section,” “device,” or “module” used herein refers to a unit for processing at least one of a function and an operation, which can be realized by hardware, software, or a combination thereof.

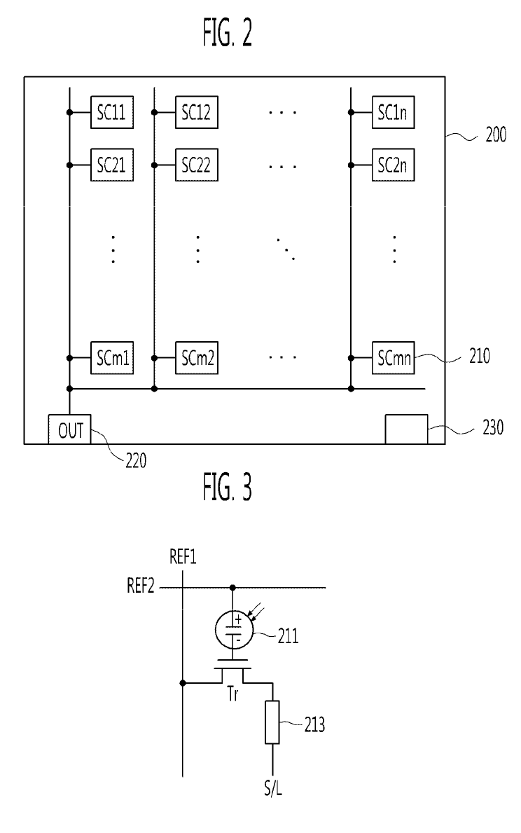

Hereinafter, a biosensor chip according to an exemplary embodiment of the present invention will be described with reference to FIG. 2

FIG. 2 illustrates the configuration of a biosensor chip according to an exemplary embodiment of the present invention.

Referring to FIG. 2, the biosensor chip according to an exemplary embodiment of the present invention includes a plurality of biosensor cells SC11 to SCmn or 210 formed on a substrate 200.

The plurality of biosensor cells SC11 to SCmn or 210 are arranged in an m-by-n matrix. Each of the biosensor cells SC11 to SCmn is formed in such a manner that a specified probe molecule is immobilized thereto.

In detail, each of the biosensor cells SC11 to SCmn includes a specified probe molecule reacting with a specified target molecule so as to allow various target molecules to be detected by one biosensor chip.

A plurality of biosensor cells SC1n to SCmn forming an n-th column are simultaneously connected with a plurality of sensing lines extending in a row direction. The plurality of sensing lines are simultaneously connected to one output terminal “OUT” or 220. Thus, the plurality of biosensor cells SC11 to SCmn arranged in a matrix output sensed signals to the outside through the output terminal “OUT.”

Unlike FIG. 2, the sensing lines may extend in a column direction, and be simultaneously connected with a plurality of biosensor cells SCm1 to SCmn forming an m-th row. The connection of the sensing lines and the biosensor cells SC11 to SCmn may be variously designed.

For example, the plurality of biosensor cells SCm1 to SCmn of the biosensor chip may be grouped in a predetermined number to form a plurality of biosensor cell groups. A plurality of biosensor cells SCm1 to SCmn of each biosensor cell group may be connected to one sensing line.

In other words, the number of biosensor cell groups may be equal to the number of sensing lines, and the number of sensing lines may be equal to the number of output terminals “OUT.”

Meanwhile, the biosensor chip includes a power supply terminal 230, which is connected with a plurality of reference voltage lines (not shown) according to a circuit of the biosensor cells SC11 to SCmn or 210 and supplies at least one reference voltage to each of the biosensor cells SC11 to SCmn or 210.

In this manner, the biosensor chip includes only the plurality of biosensor cells SC11 to SCmn or 210 arranged in a matrix without an addressing circuit for selecting at least one of the biosensor cells SC11 to SCmn or 210 intended to detect a sensed signal.

The biosensor chip is configured so that at least one of the biosensor cells SC11 to SCmn or 210 intended to detect a sensed signal is selected by external incident light, thereby outputting the sensed signal of the selected biosensor cell to the output terminal “OUT” through the sensing line connected to the selected biosensor cell.

Thus, each of the biosensor cells SC11 to SCmn or 210 includes a photoelectric element, which is selectively activated by light and outputs the sensed signal to the output terminal “OUT.”

Now, a biosensor cell according to an exemplary embodiment of the present invention will be described with reference to FIGS. 3 through 6.

FIG. 3 is a circuit diagram illustrating a biosensor cell according to a first exemplary embodiment of the present invention.

The biosensor cell according to a first exemplary embodiment of the present invention includes a plurality of biosensor cells, each of which is configured of a circuit as illustrated in FIG. 3.

Referring to FIG. 3, the biosensor cell includes a photoelectric element 211, a transistor Tr, and a biosensor 213.

The transistor Tr includes a source electrode connected with a first supply voltage REF1, a drain electrode connected to the biosensor 213, and a gate electrode connected with the photoelectric element 211.

The photoelectric element 211 is an element that causes photoelectric interaction such as a solar cell, is formed between a second supply voltage REF2 and the gate electrode of the transistor Tr, and supplies a turn-on voltage of the transistor Tr to the gate electrode in response to external light.

The biosensor 213 includes probe molecules, each of which can react with a specified target molecule. Reaction between the target molecule and the probe molecule causes a change in signal.

This probe molecule may be a material that can react with the target molecule, for instance protein, deoxyribonucleic acid (DNA) or antigen in the blood.

The biosensor 213 is connected between the drain electrode of the transistor Tr and a sensing line S/L, receives current from the drain electrode of the transistor Tr, and sends the signal, which is changed by the reaction between the target molecule and the probe molecule, to the sensing line S/L.

In this way, the photoelectric element 211 of the biosensor cell selected by the external light performs photoelectric conversion on the external light to generate an electric signal. This electric signal is supplied to the gate electrode of the transistor Tr, so that the transistor Tr is turned on, and reference current flows to the drain electrode of the transistor Tr. In other words, the biosensor 213 receives the reference current from the transistor Tr as the external light is addressed, and sends the signal changed by the reaction of its probe molecule with the target molecule, i.e. the sensed signal, to the sensing line S/L.

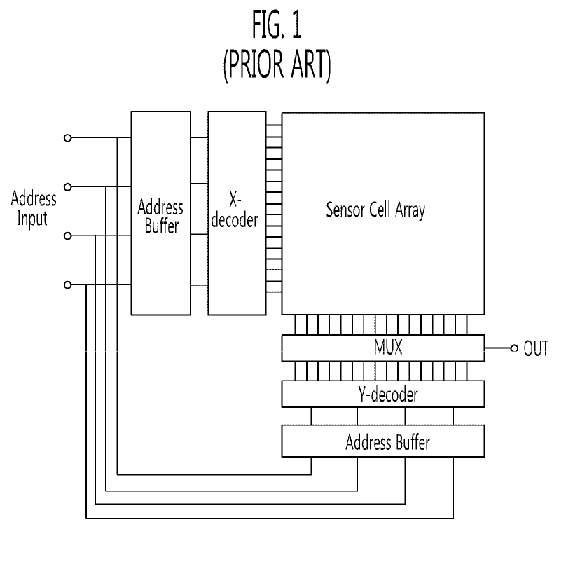

The sensed signal of the biosensor cell selected by the external light is output to the output terminal “OUT” of FIG. 1 through the sensing line S/L connected with the corresponding biosensor cell.

As such, the external light is selectively addressed to the biosensor cell, which is intended to generate and output the sensed signal using the external light, without a separate addressing circuit in the biosensor chip, so that it is possible to generate and output the sensed signal of the corresponding biosensor cell. Further, when this sensed signal is read out, it is possible to determine whether or not the target molecule reacting with the probe molecule of the corresponding biosensor cell is present in a sample.

Meanwhile, using a characteristic that the transistor Tr of the biosensor cell selected by the external light is turned on, the probe molecule may electrically undergo selective surface immobilization to a surface of the biosensor 213 of the biosensor cell.

In the selective surface immobilization using the electrical characteristic, when a voltage higher than a threshold voltage is applied to a portion for the surface immobilization, the probe molecule in a solution flowing on this portion reacts with a surface link molecule, and thus is immobilized.

Thus, when the light is addressed to the desired biosensor cell under the flow of a specified probe molecule, the transistor Tr is turned on, and thus the first supply voltage REF1 is applied to the biosensor. Thereby, the specified probe molecule currently flowing on the biosensor is selectively immobilized. Here, the first supply voltage REF1 of FIG. 3 has enough level to immobilize the specified probe molecule.

Next, when the same process is repeated in the neighboring biosensor cell under the flow of another probe molecule, the other probe molecule may be immobilized to the surface of the biosensor of the biosensor cell.

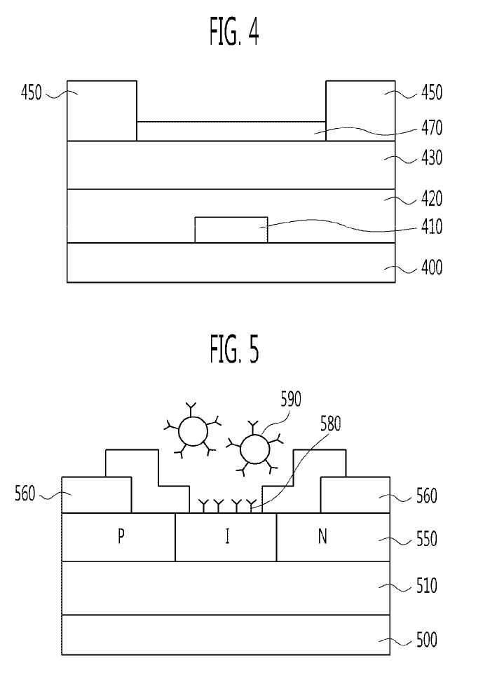

Hereinafter, another biosensor cell capable of determining whether or not a reaction occurs as light is addressed will be described with reference to FIGS. 4 through 6.

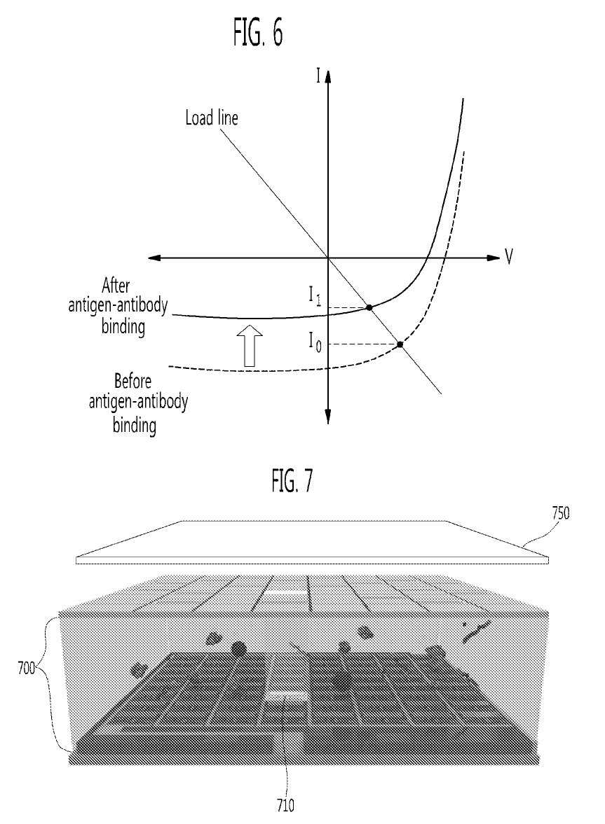

FIG. 4 is a cross-sectional view illustrating a biosensor cell according to a second exemplary embodiment of the present invention. FIG. 5 is a cross-sectional view illustrating a biosensor cell according to a third exemplary embodiment of the present invention. FIG. 6 is a graph illustrating sensed signals caused by a reaction of the biosensor cell of FIG. 5.

Referring to FIG. 4, the biosensor cell according to a second exemplary embodiment of the present invention includes a phototransistor.

The phototransistor is a combination of the transistor Tr and photoelectric element 211 of FIG. 3. In the phototransistor of FIG. 4, a source electrode 450 is connected with the first supply voltage REF1, a drain electrode 450 is connected with the biosensor 213, and a gate electrode 410 is connected with the second supply voltage REF2.

This phototransistor has a structure as illustrated in FIG. 4.

The gate electrode 410 is formed on a substrate 400. A gate insulating layer 420 and a semiconductor layer 430 are sequentially formed on the gate electrode 410.

The semiconductor layer 430 acts as a photosensitive layer, and may be doped with amorphous silicon. The semiconductor layer 430 is covered with a passivation layer 440.

This passivation layer 440 may be formed of a nitride. The source and drain electrodes 450 are formed above the gate electrode so as to be opposite to each other with the passivation layer 440 intervening therebetween.

Here, the semiconductor layer 430 has very high resistance when no external light is applied, so that the source electrode 450 is not connected with the drain electrode 450. In contrast, when the external light is applied, electron-hole pairs are generated, and thus the resistance of the semiconductor layer 430 is sharply lowered, so that the source electrode 450 is connected with the drain electrode 450.

Thus, in the case of the biosensor cell having the phototransistor of FIG. 4, when the external light is applied to the selected biosensor cell, the phototransistor of the selected biosensor cell is turned on, and thus reference current based on the first supply voltage REF1 flows to the biosensor 213 through the drain electrode 450.

The biosensor 213 receives the reference current from the phototransistor as in FIG. 3, changes a signal according to whether or not the target molecule reacts with the probe molecule, and sends the signal to the sensing line S/L.

The structure of the phototransistor of FIG. 4 is not limited to the aforementioned structure, and thus it may be formed in various shapes.

Meanwhile, the biosensor cell according to a third exemplary embodiment of the present invention may include a biosensor aligned with a photodiode as illustrated in FIG. 5.

Referring to FIG. 5, an insulating layer 510 is formed on a substrate 500, and a silicon layer 550 is formed on the insulating layer 510.

This silicon layer 550 has an n-type doping layer N, a p-type doping layer P, and a non-doping region I between the n-type doping layer N and the p-type doping layer P.

The n-type doping layer N and the p-type doping layer P may be formed by, for instance, ion implantation of the substrate 500.

Next, electrodes 560 are formed on the n-type doping layer N and the p-type doping layer P, respectively.

Each electrode 560 may be formed of a doped polysilicon layer, a metal layer, a conductive metal nitride layer, or the like. Each electrode 560 includes all materials that can be in ohmic contact with the n-type doping layer N and the p-type doping layer P.

A light absorption layer 570 is formed on this photodiode.

The light absorption layer 570 is formed to expose the non-doping region I of the silicon layer 550, and prevents external light from being transmitted in a downward direction by reflection or absorption.

The light absorption layer 570 may be formed of, for instance, metal, and be omitted.

Probe molecules 580 are immobilized on the non-doping region I exposed by the light absorption layer 570, thereby forming a biosensor.

In the biosensor cell of FIG. 5, the electrode 560 on the p-type doping layer P is connected with the supply voltage (not shown), and the electrode 560 on the n-type doping layer N is connected with the sensing line S/L.

The biosensor chip having these biosensor cells is exposed to a detection sample, thereby directing a reaction of the probe molecules of the biosensor cell with target molecules 590. The biosensor cell intended to sense the reaction is selected, and then light is addressed to the biosensor cell.

When the probe molecules of the biosensor cell to which the light is addressed react with the target molecules 590, a quantity of the light reaching the non-doping region I of the photodiode is reduced, and thus the electron-hole pairs formed in the non-doping region I are also reduced.

As such, as illustrated in FIG. 6, the current flowing between the n-type and p-type doping layers N and P of the photodiode is reduced.

When this current is output as a sensed signal to the output terminal “OUT” along the sensing line S/L, it is possible to determine whether or not the probe molecules of the corresponding biosensor cell react with the target molecules on the basis of intensity of the current.

FIG. 7 illustrates a biosensor chip and a light addressor in accordance with an exemplary embodiment of the present invention.

As illustrated in FIG. 7, the biosensor chip 700 includes a plurality of biosensor cells, each of which includes at least one specified probe molecule, without an addressing circuit.

As described above, the biosensor chip 700 is exposed to the detection sample, thereby directing a reaction of the probe molecules of the biosensor cell with target molecules. The light is selectively addressed to the biosensor cell intended to detect the reaction using the light addressor 750 outside the biosensor chip 700.

The light addressor 750 may be formed of a plurality of light sources, each of which may be selected from a short wavelength light source, a long wavelength light source, and a white light source.

A sensed signal is output to the sensing line connected with the biosensor cell selected by this addressing of the light, and then is applied to an external reading circuit through the output terminal.

Once the biosensor chip 700 including various probe molecules is exposed to the detection sample, it cannot be reused, that is, it acts as a disposable chip. The biosensor chip 700 is simplified so as to include the numerous biosensor cells and only one output terminal without the addressing circuit, and the biosensor cell intended to detect the reaction is selected by addressing the light from the light addressor, so that the biosensor chip can be manufactured by a simple process at a low cost.

According to embodiments, the biosensor cells are set in array in the biosensor chip without a separate driving unit, so that a process of manufacturing the biosensor chip is simplified. The biosensor cell to be sensed is selectively addressed through the external light, so that it is possible to reduce a price of the biosensor chip used as a disposable chip.

The exemplary embodiments of the present invention described above can be implemented not only by the apparatus and/or method, but also by a program that achieves the function corresponding to the configuration of the exemplary embodiments of the present invention or a recording medium on which the program is recorded. This will be easily implemented from the disclosure of the aforementioned exemplary embodiments of the present invention by those skilled in the art.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. A biosensor chip comprising:
a plurality of biosensor cells having:
photoelectric elements arranged in a matrix, and selectively turned on to generate a reference electric signal by addressing of external light; and
biosensors receiving the reference electric signal, and generating and outputting a sensed signal caused by a reaction between probe molecules and target molecules on a basis of the reference electric signal;
at least one sensing line simultaneously connected with the plurality of biosensor cells, and transmitting the sensed signal from one selected from the biosensor cells; and
output terminal receiving the sensed signal from the sensing line, and outputting the sensed signal to an external reader.

2. The biosensor chip according to claim 1, wherein the biosensor has resistance changed by the reaction between the probe molecules and the target molecules.

3. The biosensor chip according to claim 1, wherein the photoelectric element includes:
a solar cell generating a turn-on voltage by the addressing of the external light; and
a transistor turned on by the turn-on voltage of the solar cell and transmitting the reference electric signal to the biosensor.

4. The biosensor chip according to claim 3, wherein the transistor includes:
a gate electrode connected with the solar cell and receiving the turn-on voltage;
a source electrode connected with a reference voltage; and
a drain electrode connected with the biosensor and transmitting the reference electric signal based on the reference voltage.

5. The biosensor chip according to claim 1, wherein the photoelectric element includes a phototransistor, which is turned on by the addressing of the external light and transmits the reference electric signal to the biosensor.

6. The biosensor chip according to claim 5, wherein the phototransistor includes a semiconductor layer, from which electron-hole pairs are generated to reduce resistance by the addressing of the external light.

7. The biosensor chip according to claim 5, wherein the phototransistor includes:
a gate electrode connected with a first reference voltage a source electrode connected with a second reference voltage; and
a drain electrode connected with the biosensor and transmitting the reference electric signal based on the first and second reference voltages.

8. The biosensor chip according to claim 1, wherein:
the plurality of biosensor cells are grouped into a plurality of biosensor cell groups; and
one of the sensing lines is simultaneously connected with the plurality of biosensor cells belonging to one of the biosensor cell groups.

9. The biosensor chip according to claim 1, wherein the output terminals are equal in number to the sensing lines.

10. The biosensor chip according to claim 1, further comprising a power supply terminal, which receives an external supply voltage to apply to the plurality of biosensor cells.

11. The biosensor chip according to claim 2, wherein the probe molecules of each biosensor cell are different from each other.

12. A biosensor chip comprising:
a plurality of biosensor cells arranged in a matrix, and selectively generating and outputting a sensed signal by addressing of external light;
at least one sensing line simultaneously connected with the plurality of biosensor cells, and transmitting the sensed signal from one selected from the biosensor cells; and
an output terminal receiving the sensed signal from the sensing line, and outputting the sensed signal to an external reader.

13. The biosensor chip according to claim 12, wherein each biosensor cell includes:
a photodiode having a p-type doping layer, an n-type doping layer, and a non-doping region; and
a plurality of probe molecules immobilized on the non-doping region.

14. The biosensor chip according to claim 13, wherein the photodiode causes current to be changed by a change in transmittance caused by a reaction between the probe molecules and target molecules.

15. The biosensor chip according to claim 12, further comprising a power supply terminal, which receives an external supply voltage to apply to the plurality of biosensor cells.

16. The biosensor chip according to claim 13, wherein the probe molecules of each biosensor cell are different from each other.

17. A method of driving a biosensor chip comprising:
exposing a plurality of biosensor cells arranged in a matrix to a detection sample having target molecules;
selecting at least one of the biosensor cells which is intended to generate and output a sensed signal, and addressing external light to the selected biosensor cell:
turning on a transistor of the selected biosensor cell with the light, and outputting a reference electric signal to a biosensor of the selected biosensor cell; and
generating the sensed signal from the biosensor based on the reference electric signal, and outputting the sensed signal to an output terminal.

18. The method according to claim 17, wherein the sensed signal of the biosensor cell is transmitted to the output terminal through a sensing line connected with the plurality of biosensor cells at the same time.

19. The method according to claim 18, wherein each biosensor cell includes:
a solar cell generating a turn-on voltage with the external light;
a transistor turned on by the turn-on voltage of the solar cell and transmitting the reference electric signal; and
the biosensor receiving the reference electric signal of the transistor to generate the sensed signal changed by a reaction between probe molecules and target molecules.

20. The method according to claim 18, wherein each biosensor cell includes:
a phototransistor having a semiconductor layer from which electron-hole pairs are generated by the external light to reduce resistance, turned on by the external light, and transmitting the reference electric signal; and
the biosensor receiving the reference electric signal of the phototransistor to generate the sensed signal changed by a reaction between probe molecules and target molecules.
